(19) United States
(12) Patent Application Publication
(10) Pub. No.: US 2023/0382887 A1
(43) Pub. Date: Nov. 30, 2023

(54) PROCESS FOR THE PREPARATION OF CHLORANTRANILIPROLE

(71) Applicant: Gharda Chemicals Limited, Maharashtra (IN)

(72) Inventors: Suchet Saran MATHUR, Thane, Maharashtra (IN); Hridaynath Vishwanath MHATRE, Thane, Maharashtra (IN); Vishal Parshuram PEDHAVI, Thane, Maharashtra (IN); Uday Tukaram KOLI, Thane, Maharashtra (IN)

(21) Appl. No.: 18/032,706

(22) PCT Filed: Oct. 20, 2021
(86) PCT No.: PCT/IB2021/059683

§ 371 Date: Apr. 19, 2023

(30) Foreign Application Priority Data
Oct. 20, 2020 (IN) 202021045725

(51) Int. Cl. C07D 401/04

(52) U.S. Cl. CPC C07D 401/04

(57) ABSTRACT

A process for the preparation of chlorantraniliprole is provided. The process is carried out at an ambient temperature by using an inorganic base which can be easily separated. The process is simple, efficient, environment friendly, and provides chlorantraniliprole with high purity and high yield.

### FIELD

The present disclosure relates to a process for the preparation of Chlorantraniliprole.

### BACKGROUND

The background information herein below relates to the present disclosure but is not necessarily prior art.

Chlorantraniliprole is a broad-spectrum anthranilic diamide insecticide. Chlorantraniliprole, when consumed by insects, it interrupts the normal muscle contraction, resulting in the death of the insects. The structural formula for Chlorantraniliprole is as given below.

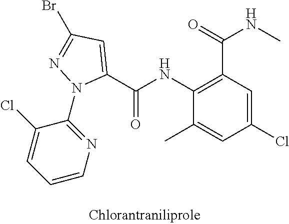 [SMILES: CNC(=O)c1cc(Cl)cc(C)c1NC(=O)c1cc(Br)nn1-c1ncccc1Cl]

Various methods for the preparation of Chlorantraniliprole are known in the art, however, the conventional methods employ expensive and toxic organic solvents and catalysts for the reactions, which makes the process costly as well as harmful to the environment. Further, the conventional methods of preparing Chlorantraniliprole require an excessive amount of methane sulfonyl chloride, which leads to the production of a large amount of highly polluting sulfur-containing organic acid wastewater which is difficult to treat. In addition, these methods lead to a low yield of Chlorantraniliprole and hence are unsuitable for industrial production.

Therefore, there is felt a need to provide a process for the preparation of Chlorantraniliprole that mitigates the aforestated drawbacks.

### Objects

Some of the objects of the present disclosure, which at least one embodiment herein satisfies, are as follows:

It is an object of the present disclosure to ameliorate one or more problems of the prior art or to at least provide a useful alternative.

Another object of the present disclosure is to provide a process for the preparation of Chlorantraniliprole.

Still another object of the present disclosure is to provide a process for the preparation of Chlorantraniliprole which is simple, efficient, and environment friendly.

Other objects and advantages of the present disclosure will be more apparent from the following description, which is not intended to limit the scope of the present disclosure.

### SUMMARY

The present disclosure relates to a process for preparation of Chlorantraniliprole. The process comprises reacting 3-bromo-1-(3-chloro-2-pyridinyl)-1H-pyrazole-5-carboxylic acid with 2-amino-5-chloro-3-methylbenzoic acid in a first fluid medium by using at least one inorganic base and methane sulfonyl chloride under stirring at a first predetermined temperature for a first predetermined time period to obtain a benzoxazinone intermediate. The benzoxazinone intermediate is then reacted with an organic amine to obtain Chlorantraniliprole of formula (I). The inorganic base and the methane sulfonyl chloride used during the process is added in portions.

### DETAILED DESCRIPTION

Embodiments, of the present disclosure, will now be described herein. Embodiments are provided so as to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open ended transitional phrases and therefore specify the presence of stated features, integers, steps, operations, elements, modules, units and/or components, but do not forbid the presence or addition of one or more other features, integers, steps, operations, elements, components, and/or groups thereof. The particular order of steps disclosed in the method and process of the present disclosure is not to be construed as necessarily requiring their performance as described or illustrated. It is also to be understood that additional or alternative steps may be employed.

As used herein, the term “and/or” includes any and all combinations of one or more of the associated listed elements.

The terms first, second, third, etc., should not be construed to limit the scope of the present disclosure as the aforementioned terms may be only used to distinguish one element, component, region, layer or section from another component, region, layer or section. Terms such as first, second, third etc., when used herein do not imply a specific sequence or order unless clearly suggested by the present disclosure.

Chlorantraniliprole is an insecticide of the ryanoid class. Chlorantraniliprole belongs to a new class of selective insecticides featuring a novel mode of action to control a range of pests.

Various methods for the preparation of Chlorantraniliprole are reported. However, the conventional methods of preparing Chlorantraniliprole require highly toxic reagents such as methyl chloroformate and phosgene, resulting in operational inconveniences and unsuitability for large scale production.

Chlorantraniliprole is represented by the following structure:

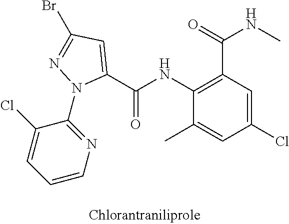 [SMILES: CNC(=O)c1cc(Cl)cc(C)c1NC(=O)c1cc(Br)nn1-c1ncccc1Cl]

Chemical Name: 3-Bromo-N-[4-chloro-2-methyl-6-[(methylamino)carbonyl]phenyl]-1-(3-chloro-2-pyridinyl)-1H-pyrazole-5-carboxamide

CAS No.: 500008-45-7

The process of the present disclosure provides a simple, environment friendly, and economical process that results in improved yields and higher purity of the final product.

The process is described in detail.

In a first step, 3-bromo-1-(3-chloro-2-pyridinyl)-1H-pyrazole-5-carboxylic acid is reacted with 2-amino-5-chloro-3-methylbenzoic acid in a first fluid medium by using at least one inorganic base and methane sulfonyl chloride under stirring at a first predetermined temperature for a first predetermined time period to obtain a benzoxazinone intermediate (2-[5-Bromo-2-(3-chloro-pyridin-2-yl)-2H-pyrazol-3-yl]-6-chloro-8-methyl-benzo[d][1,3]oxazin-4-one).

In accordance with the present disclosure, the first fluid medium is selected from the group consisting of acetonitrile, methylene dichloride, methyl ethyl ketone, methanol, ethanol, n-propanol, isopropanol, n-butanol, iso-butanol, and tert-butanol. In an exemplary embodiment of the present disclosure, the first fluid medium is acetonitrile.

In accordance with the present disclosure, the inorganic base is selected from potassium carbonate, potassium bicarbonate, sodium carbonate, sodium bicarbonate, potassium hydroxide, sodium hydroxide, lithium carbonate, lithium hydroxide monohydrate, magnesium carbonate, magnesium hydroxide, calcium carbonate and calcium hydroxide.

In an exemplary embodiment of the present disclosure, the inorganic base is potassium carbonate. In another exemplary embodiment of the present disclosure, the inorganic base is potassium bicarbonate. In yet another exemplary embodiment of the present disclosure, the inorganic base is sodium carbonate. In still another exemplary embodiment of the present disclosure, the inorganic base is sodium bicarbonate.

In accordance with the present disclosure, the first predetermined temperature is in the range of 20° C. to 50° C. In an exemplary embodiment of the present disclosure, the first predetermined temperature is 30° C. In another exemplary embodiment of the present disclosure, the first predetermined temperature is 28° C.

In accordance with the present disclosure, the first predetermined time period is in the range of 1 hour to 10 hours. In an exemplary embodiment of the present disclosure, the first predetermined time period of 6 hours.

In accordance with the present disclosure, the ratio of the inorganic base to the 3-bromo-1-(3-chloro-2-pyridinyl)-1H-pyrazole-5-carboxylic acid is in the range of 0.5:1 to 1.5:1 In an exemplary embodiment of the present disclosure, the ratio of the inorganic base to the 3-bromo-1-(3-chloro-2-pyridinyl)-1H-pyrazole-5-carboxylic acid is 0.8:1. In another exemplary embodiment of the present disclosure, the ratio of the inorganic base to the 3-bromo-1-(3-chloro-2-pyridinyl)-1H-pyrazole-5-carboxylic acid is 0.9:1. In still another exemplary embodiment of the present disclosure, the ratio of the inorganic base to the 3-bromo-1-(3-chloro-2-pyridinyl)-1H-pyrazole-5-carboxylic acid is 1.3:1.

In a second step, the so obtained benzoxazinone intermediate is reacted with an organic amine under stirring to obtain a product mass containing Chlorantraniliprole of formula (I).

In accordance with the present disclosure, the organic amine is methylamine.

In accordance with the present disclosure, the ratio of 2-amino-5-chloro-3-methylbenzoic acid to the methane sulfonyl chloride is in the range of 0.5:1 to 1:1. In an exemplary embodiment of the present disclosure, the ratio of 2-amino-5-chloro-3-methylbenzoic acid to the methane sulfonyl chloride is 0.8:1.

In accordance with the embodiment of the present disclosure, the benzoxazinone intermediate is purified, and the purified benzoxazinone intermediate is reacted with organic amine in a second fluid medium at a second predetermined temperature for a second predetermined time period to obtain Chlorantraniliprole.

In accordance with the present disclosure, the second predetermined temperature is in the range of 25 to 50° C. In an exemplary embodiment of the present disclosure, the second predetermined temperature is 32° C. In another exemplary embodiment of the present disclosure, the second predetermined temperature is 35° C.

In accordance with the present disclosure, the second predetermined time period is in the range of 8 to 12 hours. In an exemplary embodiment of the present disclosure, the second predetermined time period is 10 hours.

In accordance with the present disclosure, the second fluid medium is selected from acetonitrile and isopropyl alcohol.

In accordance with the present disclosure, the inorganic base and the methane sulfonyl chloride are added in portions.

In an exemplary embodiment of the present disclosure, the second fluid medium is isopropyl alcohol. In another exemplary embodiment of the present disclosure, the second fluid medium is acetonitrile.

In accordance with the present disclosure, the methane sulfonyl chloride is separated, recovered, and recycled during the process.

In accordance with the present disclosure, the so obtained product mass containing Chlorantraniliprole of formula (I) is filtered and washed with the first fluid medium to obtain a cake. The cake is added into water under stirring to obtain a slurry. The slurry is filtered, washed with water, and suction dried to obtain Chlorantraniliprole of formula (I).

In accordance with the present disclosure, the process for the preparation of Chlorantraniliprole is carried out in a single step i.e., without purifying the benzoxazinone intermediate, or in two steps i.e., by purifying the benzoxazinone intermediate.

In accordance with the present disclosure, optionally the benzoxazinone intermediate is purified via filtration process followed by washing with acetonitrile, water, and drying.

In an exemplary embodiment, the schematic representation of the preparation of Chlorantraniliprole in accordance with the present disclosure is given below as Scheme 1

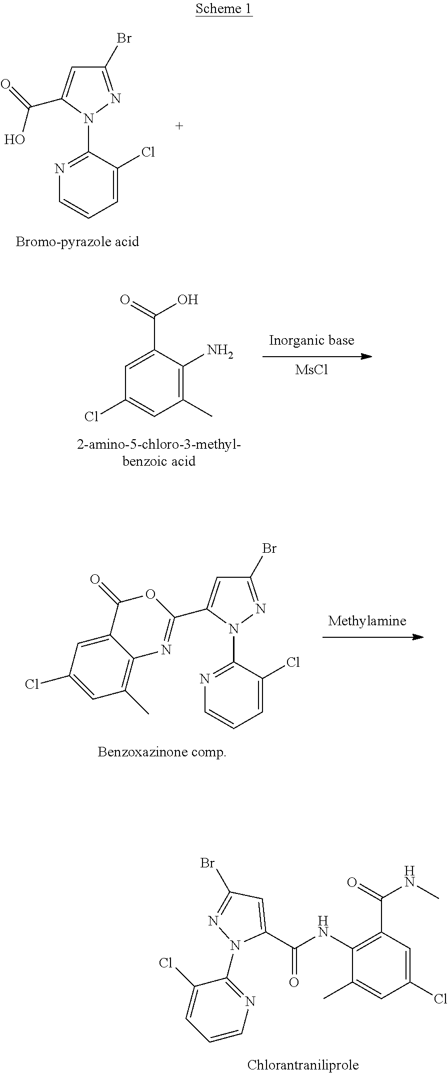 [SMILES: "MsCl" ; O=C(O)c1cc(Br)nn1-c1ncccc1Cl ; Cc1cc(Cl)cc(C(=O)O)c1N ; Cc1cc(Cl)cc2c(=O)oc(-c3cc(Br)nn3-c3ncccc3Cl)nc12 ; CNC(=O)c1cc(Cl)cc(C)c1NC(=O)c1cc(Br)nn1-c1ncccc1Cl]

The present disclosure provides a simple process for the preparation of Chlorantraniliprole which provides a comparatively higher yield of the product (Chlorantraniliprole) with greater purity.

The process of the present disclosure employs inorganic bases which are cheaper than organic bases such as pyridine, picolines, and the like; and therefore, the process of the present disclosure is cost-efficient and economical. The inorganic bases used in the process of the present disclosure can be easily separated making the process environment friendly.

The process of the present disclosure is carried out at ambient temperatures. Thus, the process of the present disclosure is energy efficient.

The foregoing description of the embodiments has been provided for purposes of illustration and not intended to limit the scope of the present disclosure. Individual components of a particular embodiment are generally not limited to that particular embodiment, but, are interchangeable. Such variations are not to be regarded as a departure from the present disclosure, and all such modifications are considered to be within the scope of the present disclosure.

The present disclosure is further described in light of the following experiments which are set forth for illustration purpose only and not to be construed for limiting the scope of the disclosure. The following experiments are scalable to industrial/commercial process.

### Experimental Details

### Example 1

### Preparation of Chlorantraniliprole by Using Potassium Carbonate (K₂CO₃) as an Inorganic Base (in Two Steps)

### Step(i):

200 ml of acetonitrile was added in a reactor followed by the addition of 31 gms of 3-Bromo-1-(3-chloro-2-pyridinyl)-1H-pyrazole-5-carboxylic acid under stirring to obtain a mixture. 15 gms of potassium carbonate (first portion) was added to the mixture and equilibrated at 28° C. for 15 min followed by adding 12 gms of methane sulfonyl chloride (MsCl) (first portion) under stirring at 28° C. for 30 minutes to obtain a slurry. 19 gms of 2-amino-5-chloro-3-methylbenzoic acid was added to the so obtained slurry under stirring followed by adding 15 gms of potassium carbonate (second portion) and 12 gms of MsCl (second portion) and further stirred at 30° C. for 6 hrs to obtain a mass containing 2-[5-Bromo-2-(3-chloro-pyridin-2-yl)-2H-pyrazol-3-yl]-6-chloro-8-methyl-benzo[d][1,3]oxazin-4-one (Benzoxazinone intermediate).

The mass was filtered at 30° C. and was washed with acetonitrile to obtain a cake. The cake was reslurried in water at 30° C. and again filtered to obtain a residue. The residue was washed with water and solids so obtained was suction dried to obtain Benzoxazinone intermediate having a HPLC purity of 96%, and the yield on purity 72%.

### Step (ii):

34 gms of Benzoxazinone intermediate obtained in step (i) was added in 75 ml of aqueous isopropyl alcohol (IPA) followed by adding 9 gms of 40% aqueous methylamine and stirred at 32° C. for 10 hours to obtain a product mass containing Chlorantraniliprole having an HPLC purity of 95% and the yield on purity was 61%.

### Example 2

### Preparation of Chlorantraniliprole by Using Sodium Carbonate (Na₂CO₃) as an Inorganic Base (in Two Steps)

### Step-(i):

400 ml of acetonitrile was added in a reactor followed by the addition of 48 gms of 3-Bromo-1-(3-chloro-2-pyridinyl)-1H-pyrazole-5-carboxylic acid to obtain a mixture. 19.2 gms of sodium carbonate (first portion) and 18.2 gms of MsCl (first portion) were added to the mixture under stirring at 28° C. for 30 minutes to obtain a slurry. 28.2 gems of 2-amino-5-chloro-3-methylbenzoic acid was added to the so obtained slurry under stirring followed by adding 19.2 gms of sodium carbonate (second portion) and 18.2gms of MsCl (second portion) under stirring at 30° C. for 4 hours to obtain a mass containing 2-[5-Bromo-2-(3-chloro-pyridin-2-yl)-2H-pyrazol-3-yl]-6-chloro-8-methyl-benzo[d][1,3]oxazin-4one (Benzoxazinone intermediate).

The mass was filtered at 30° C. and was washed with acetonitrile to obtain a cake. The cake was reslurried in water at 30° C. and again filtered to obtain a residue. The residue was washed with water and solids so obtained was suction dried to obtain Benzoxazinone intermediate having a HPLC purity of 96.98%, and the yield on purity 90%.

### Step-(ii):

63 gms of Benzoxazinone intermediate obtained in step (i) was added in 300 ml of acetonitrile followed by passing methylamine gas and was stirred at 35° C. for 10 hours to obtain a product mass containing Chlorantraniliprole having an HPLC purity of 96.86% and the yield on purity was 80%.

### Example 3

### Preparation of Chlorantraniliprole by Using Na₂CO₃ as an Inorganic Base (in a Single Step)

400 ml of acetonitrile was added in a reactor followed by the addition of 48 gms of 3-Bromo-1-(3-chloro-2-pyridinyl)-1H-pyrazole-5-carboxylic acid under stirring at 25° C. to obtain a mixture. 19 gms of sodium carbonate (first portion) was added at 28° C. to the mixture for 15 min followed by adding 19 gms of MsCl (first portion) under stirring at 32° C. for 30 mins to obtain a slurry. 32 gms of 2-amino-5-chloro-3-methylbenzoic acid was added to the slurry under stirring for 30 min followed by adding 19 gms of sodium carbonate (second portion) for 15 min and 19 gms of MsCl (second portion) for 1 hour and further stirred at 30° C. for 6 hrs to obtain a mass containing of 2-[5-Bromo-2-(3-chloro-pyridin-2-yl)-2H-pyrazol-3-yl]-6-chloro-8-methyl-benzo[d][1,3]oxazin-4one(Benzoxazinone intermediate). Methylamine gas was passed through the mass till HPLC showed the absence of the benzoxazinone intermediate to obtain a product mass containing Chlorantraniliprole.

The product mass was filtered and was washed with acetonitrile to obtain a cake. The cake was reslurried in water at 30° C. followed by filtration to obtain a residue. The residue was washed with water and solids so obtained were suction dried to obtain a purified Chlorantraniliprole having an HPLC purity of 95.5% and the yield on purity was 85%.

### Example 4

### Preparation of Chlorantraniliprole by Using KHCO 3 as an Inorganic Base (in a Single Step)

250 ml of acetonitrile was added in a reactor followed by the addition of 32 gms of 3-Bromo-1-(3-chloro-2-pyridinyl)-1H-pyrazole-5-carboxylic acid under stirring at 25° C. to obtain a mixture. 21 gms of potassium bicarbonate (first portion) was added to the mixture for 15 min followed by adding 12 gms of MsCl (first portion) for 30 min under stirring and further equilibrated at 25° C. for 30 mins to obtain a slurry. 19 gms of 2-amino-5-chloro-3-methylbenzoic acid was added to the so obtained slurry under stirring and maintained at 28° C. to 32° C. for 30 min to obtain a reaction mixture. 21 gms of potassium bicarbonate (second portion) was added to the reaction mixture for 15 min followed by adding 12 gms of MsCl (second portion) for 30 min and equilibrated at 28 to 32° C. for 6 hours to obtain a mass containing 2-[5-Bromo-2-(3-chloro-pyridin-2-yl)-2H-pyrazol-3-yl]-6-chloro-8-methyl-benzo[d][1,3 ]oxazin-4one(Benzoxazinone intermediate).

Methylamine gas was passed through the mass till HPLC showed the absence of the benzoxazinone intermediate to obtain a product mass containing Chlorantraniliprole.

The product mass was filtered and was washed with acetonitrile to obtain a wet cake. The wet cake was reslurried in water and again filtered to obtain a residue. The residue was washed with water and solids obtained were suction dried to obtain a purified Chlorantraniliprole having an HPLC purity of 97% and the yield on purity was 78%.

### Example 5

### Preparation of Chlorantraniliprole by Using NaHCO₃ as an Inorganic Base (in a Single Step)

250 ml of acetonitrile was added in a reactor followed by the addition of 31 gms of 3-Bromo-1-(3-chloro-2-pyridinyl)-1H-pyrazole-5-carboxylic acid under stirring at 25° C. to obtain a mixture. 17.6 gms of sodium bicarbonate (first portion) was added to the mixture for 15 min followed by adding 12 gms of MsCl (first portion) for 30 min under stirring and further equilibrated at 25° C. for 30 mins to obtain a slurry. 19 gms of 2-amino-5-chloro-3-methylbenzoic acid was added to the so obtained slurry under stirring and maintained at 28° C. to 32° C. for 30 min to obtain a reaction mixture. 17.6 gms of potassium bicarbonate (second portion) was added to the reaction mixture for 15 min followed by adding 12 gms of MsCl (second portion) for 30 min and equilibrated at 28 to 32° C. for 6 hours to obtain a mass containing 2-[5-Bromo-2-(3-chloro-pyridin-2-yl)-2H-pyrazol-3-yl]-6-chloro-8-methyl-benzo[d][1,3 ]oxazin-4one(Benzoxazinone intermediate).

Methylamine gas was passed through the mass till HPLC showed the absence of the benzoxazinone intermediate to obtain a product mass containing Chlorantraniliprole.

The product mass was filtered and was washed with acetonitrile to obtain a wet cake. The wet cake was reslurried in water and again filtered to obtain a residue. The residue was washed with water and solids obtained were suction dried to obtain a purified Chlorantraniliprole having an HPLC purity of 97% and the yield on purity was 82%.

### Technical Advancement

The present disclosure described herein above has several technical advantages including, but not limited to, the realization of a process for the preparation of Chlorantraniliprole which:
is simple, economical, efficient, commercially scalable, and environment friendly;
results in higher yield and purity of the product (Chlorantraniliprole);
the fluid medium and methane sulfonyl chloride can be separated, recovered, and recycled.
employs relatively cheaper and non-hazardous reagents; and
the inorganic base used in the process is inexpensive and can be easily separated.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The use of the expression “at least” or “at least one” suggests the use of one or more elements or ingredients or quantities, as the use may be in the embodiment of the invention to achieve one or more of the desired objects or results. While certain embodiments of the inventions have been described, these embodiments have been presented by way of example only, and are not intended to limit the scope of the inventions. Variations or modifications to the formulation of this invention, within the scope of the invention, may occur to those skilled in the art upon reviewing the disclosure herein. Such variations or modifications are well within the spirit of this invention.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values given for various physical parameters, dimensions, and quantities are only approximate values and it is envisaged that the values higher than the numerical value assigned to the physical parameters, dimensions and quantities fall within the scope of the invention unless there is a statement in the specification to the contrary.

While considerable emphasis has been placed herein on the specific features of the preferred embodiment, it will be appreciated that many additional features can be added and that many changes can be made in the preferred embodiment without departing from the principles of the disclosure. These and other changes in the preferred embodiment of the disclosure will be apparent to those skilled in the art from the disclosure herein, whereby it is to be distinctly understood that the foregoing descriptive matter is to be interpreted merely as illustrative of the disclosure and not as a limitation.

1. A process for the preparation of Chlorantraniliprole represented by formula (I): 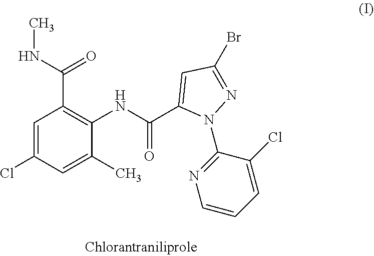 [SMILES: CNC(=O)c1cc(Cl)cc(C)c1NC(=O)c1cc(Br)nn1-c1ncccc1Cl]
said process comprising the following steps:
i. reacting 3-bromo-1-(3-chloro-2-pyridinyl)-1H-pyrazole-5-carboxylic acid with 2-amino-5-chloro-3-methylbenzoic acid in a first fluid medium by using at least one inorganic base and methane sulfonyl chloride under stirring at a first predetermined temperature for a first predetermined time period to obtain a benzoxazinone intermediate; and
ii. reacting the benzoxazinone intermediate with an organic amine under stirring to obtain the Chlorantraniliprole of formula (I).

2. The process as claimed in claim 1, wherein said first fluid medium is at least one selected from the group consisting of acetonitrile, methylene dichloride, methyl ethyl ketone, methanol, ethanol, n-propanol, isopropanol, n-butanol, iso-butanol, and tert-butanol.

3. The process as claimed in claim 2, wherein said first fluid medium is acetonitrile.

4. The process as claimed in claim 1, wherein said inorganic base is selected from the group consisting of potassium carbonate, sodium carbonate, sodium bicarbonate, potassium bicarbonate, sodium hydroxide, potassium hydroxide, magnesium carbonate, magnesium hydroxide, lithium carbonate, lithium hydroxide monohydrate, cesium carbonate, calcium carbonate, and calcium hydroxide.

5. The process as claimed in claim 1, wherein said first predetermined temperature is in the range of 20° C. to 50° C. and wherein said first predetermined time period is in the range of 1 hour to 10 hours.

6. The process as claimed in claim 1, wherein said organic amine is methylamine.

7. The process as claimed in claim 1, wherein said benzoxazinone intermediate of step (i) is purified and said purified benzoxazinone intermediate is reacted with said organic amine in a second fluid medium at a second predetermined temperature for a second predetermined time period to obtain Chlorantraniliprole.

8. The process as claimed in claim 7, wherein said second predetermined temperature is in the range of 25° C. to 50° C. and said second predetermined time period is in the range of 8 hours to 12 hours.

9. The process as claimed in claim 7, wherein said second fluid medium is selected from isopropyl alcohol and acetonitrile.

10. The process as claimed in claim 1, wherein said inorganic base and said methane sulfonyl chloride are added in portions.

11. The process as claimed in claim 1, wherein a weight ratio of said inorganic base to said 3-bromo-1-(3-chloro-2-pyridinyl)-1H-pyrazole-5-carboxylic acid is in the range of 0.5:1 to 1.5:1.

12. The process as claimed in claim 1, wherein a weight ratio of said 2-amino-5-chloro-3-methylbenzoic acid to said methane sulfonyl chloride is in the range of 0.5:1 to 1:1.

13. The process as claimed in claim 1, wherein said methane sulfonyl chloride used in step-(i) is separated, recovered, and recycled.
